(19) United States
(12) Patent Application Publication
(10) Pub. No.: US 2007/0239207 A1
(43) Pub. Date: Oct. 11, 2007

(54) Surgery thread

(76) Inventor: Jose Antonio Encinas Beramendi, Rio de Janeiro (BR)

Correspondence Address: Finnegan Henderson Farabow;Garrett & Dunner, 901 New York Avenue N W, Washington, DC, 20001-4413, US

(21) Appl. No.: 11/632,316

(22) PCT Filed: Feb. 15, 2005
(86) PCT No.: PCT/BR05/00019

§ 371 Date: Jan. 12, 2007

(30) Foreign Application Priority Data
Jul. 13, 2004 (BR) PI0402762-0
Sep. 20, 2004 (BR) C10402762-0

(51) Int. Cl. A61L 17/00

(52) U.S. Cl. 606/228

(57) ABSTRACT

The present invention refers to surgery threads which are flexible, made of resistant inert plastic material and being employed in aesthetic surgeries in the cases of skin ageing prevention and/or correction surgeries for motion injury signals, wherein the threads comprise a previous defined sequence of a tissue support arrangement having a number of fixing claws placed alongside of the thread surface and wherein an arrangement of an upstream claw (1) set and a downstream claw (2) set is provided.

The present invention refers to surgery threads which are flexible, made of resistant inert plastic material and being employed in aesthetic surgeries in the cases of skin ageing prevention and/or correction surgeries for motion injury signals.

### BACKGROUND OF THE INVENTION

The Medical Progress is to be seen as a very remarkable development when considered the necessary time for its accomplishment. Some diseases and surgery limitations, critical problems in the past, are now easily overcame, therefore, the Health quality and lifetime of the Human being have been increased due to the Medical advance but, much more of such Progress is to be reached.

One of the most developed Medical field comprises the Aesthetical/Corrective Surgery. Aesthetical surgeries comprise a set of medical procedures for correct ageing signals or physical complaints which correspond to a physical limitation or a Social barrier to individuals. Many physical complaints are scar type and they tend to lower the self respect of the patient and may cause social rejection and social stigmatizing.

Most of such physical complaints are caused by mechanical origin accidents such as falling, shock and several physical kinds of trauma. In some cases, physical complaints have genetic or ever acquired origin; the former requests surgical correction as in the cleft lips; the latter comprises the most common case, for example, brain injury can cause movement limitations, loss of the local muscular control and local degeneration as in facial paralysis or paraplegia. The loss of the motion ability causes the muscular atrophy and the body shape tend to be assynmetrical; Although the physical restoring is considered an important part of the patient healing in the cases of physical complaint, emotional support for self respect restoration is also a valuable part in the patient healing program. Corrective surgeries can provide physical restoration and emotional support to the patient.

The synmetry restoration of the damaged area offers to the patient a feeling of comfort and redution of the lesion effects. This feeling is important for the complementary therapy success applied during the patient healing.

Other important contribution of the Aesthetical/Correction surgeries is the ageing signals prevention. Some individuals tend to acquire more ageing signals than the others and it comprise the enhancing of the face signals such as facial foldings and flaccid skin, in many situations such signals appear before the due time. Therefore, as in the above discussed processes, the degeneration process (natural or not) influence the emotional balance; the self respect of an individual is crucial for the maintenance of the metabolic process, particularly the immunological system at acceptable levels.

The physical degradation sensation has a strong effect in the regulation functions of the body metabolism; In many cases one can notice increasing of infectious diseases or others caused by the continous lower of the self respect of an individual when he strongly believes in a quick body degradation and so permits a desease raising.

Once more, the reduction of such signals provide an enhancing of the self respect levels of a patient and avoid that an effective body degradation process be started just the ageing became.

Several surgery approaches are known in the art. For ageing cases, local teraphy based on bacterial toxins, particularly the botulism bacterial toxins, having a skin hardening effect provide a kind of self support of the flaccid tissue and a less aged/tired look. Also some injection based fill-treatment in which collagen/silicone containing solution (Medical quality) are injected into the skin folds caused by the ageing process and the results are partially satisfactory. In these cases, additional treatments are periodically necessary and the term of the additional treatment is considerably short and the cost of it is considerably high. Further, many patients complains of the pain caused by the injection treatment in more sensitive parts, as in the sinus bone, or around the eyeball cavity.

Other embodiments comprise prosthesis treatments. Such prosthesis are very common in the cases of breast surgery ou in injuries located in the face or in the rump. The approaches of such procedure are very known in the Medical Art. However, such approaches are extremely invasive, contribute for surgical shock situations and the healing time is considerably large. Another aspect related to the referred approaches comprises periodical inspection and the risk of body rejection; furthermore the aesthetic effect is quite dependent of the material quality, i.e., the prosthesis instrument can be misadjusted the local body part and the effect will be not satisfactory.

Another embodiment comprise surgical threads for flaccid tissue lifting; such approach is known as surgical thread lifting. The surgical thread is applied under the skin through the fat tissue performing a cohesive lifting effect. One advantageous feature of the above mentioned approach is that it is less invasive than the above mentioned invasive alternatives, reducing the not desired effects previously discussed; the surgical threads—inserted under the sking in the fat tissue—permit a better positioning during the correction surgery providing an enhanced aesthetic effect; the tensile strength and the length of the thread can be defined by the surgeon.

However, the surgical threads of the art has a fragile tensile strength when they are submitted to mechanical tension, therefore the resulting effect is not satisfactory and another correction procedure will be necessary. Another limiting aspect of the art comprise the fragility of the hooks. Threads of the art have hooks which is inclined in relation to the thread axis and it comprises a fragile point of the structure. The hooks of such known threads commonly collapses in a progressive effect, therefore a loss of tissue lifting feature is verified.

Some examples of the art are presently cited as follows:

The patent document WO02004006086 refers to a lifting strap for flaccid tissues—“endoprosthesis”—comprising an inert material strap in which in the strap surface a set of arrow point type hooks are provided for support the flaccid is retained and supported; as seen in the referred document, the “endoprosthesis” is directed to breast and/or nose correction procedures. The “endoprosthesis” strap comprises a piece having large size which causes a big sliding under the skin, i.e., in the case in that the patient moves, all this piece would move producing an under-skin-movement and a non aesthetic, non natural movement.

The patent document WO03103733 refers to a thread for correction surgery which comprises inclined hooks having conical shape and such hooks are placed in series alongside the thread axis and the edge of the hooks are sharpened and flexible. The hooks are suitably placed in the thread surface (laterally or in the whole axis). The referred thread has a monotone hook arrangement in over the cilindrical body of the thread axis causing a poor aesthetic effect even such effect show some effectiveness. The lifting effect of this invention causes a loss of free movement which is responsible for a natural movement effect, therefore the advantageous feature of the mechanical resistance of the hooks arrangement is not reached. As in the previous discussed document, the hooks are undully described as being conical, however, the Figures of the referred invention denies such conical shape; both in the strap and in the thread discussed above the hooks comprises are limited to planar projections having an arrow point shape.

The patent document EP1075843 refers to a surgical thread for flaccid tissue lifting in which hooks placed alongside the thread body are sequentially oriented and in an opposed direction in relation to the thread traction sense. A first hook arrangement is placed in a direction while the second arrangement is placed in the opposed direction for avoid displacement of the thread under the skin. Also, is provided a method for manufacturing a thread which is able to be inserted in a single thread direction. As in the previous case, the most enhanced is the lifting effect, the less enhanced is the natural movement effect.

The document U.S. Pat. No. 5,584,859 refers to a surgery thread which is biologically absorbed by the organism and have a set of hooks placed in the corresponding cilindrical body and a central member from which a network is built as a fabric for permit tissues be united during the cicatrix process. Even the present surgical thread has a similar shape in relation to the previously discussed cases, such thread is directed to a particular end: to favor the cicatrix procedure and after this procedure the thread is absorbed by the organism.

### OBJECTIVE OF THE INVENTION

One objective of the present invention is to provide a solution for the above discussed problems. In this sense, the present invention refers to a aesthetic/correction surgery thread comprising a cilindrical body on which a set of claws are placed in a particular arrangement which is suitable to correction and/or lifting of a certain body tissue, for preserving the natural free movement of the lifted part.

### DESCRIPTION OF THE DRAWINGS

FIG. 1 is a schematic illustration of one embodiment of the present invention.

FIG. 2 is a schematic illustration of another embodiment of the present invention.

FIG. 3 is a schematic illustration of another embodiment of the present invention.

FIG. 4 is a schematic illustration of another embodiment of the present invention.

FIGS. 5A-5E illustrate schemes of an aesthetic facial lifting.

FIGS. 6A-6B illustrate schemes of lifting thread application in facial paralysis correction surgeries.

FIGS. 7A-7B illustrate schemes of lifting thread application in flaccid tissue lifting surgeries in the Human neck.

FIGS. 8A-8B illustrate schemes for lifting thread application in arm diastasis and umbilucus lifting.

FIG. 9 illustrate another embodiment of surgical thread according to the present invention.

FIG. 10 illustrate a detailed view of the embodiment of surgical thread of FIG. 9.

FIG. 11 illustrate another embodiment of surgical thread according to the present invention.

FIG. 12 illustrate a detailed view of the embodiment of surgical thread of FIG. 11.

FIG. 13 illustrate a top view of a surgical thread moulding plate according to the present invention.

FIG. 14 illustrate a perspective view of the surgical thread moulding box according to the present invention.

### DETAILED DESCRIPTION OF THE PREFERRED EMBODIMENTS

The present invention refers to a surgical threads which are indicated to flaccid tissue lifting for permit that a patient has a natural aspect in Human body parts damaged by movement paralysis ou by ageing process.

For a better effect of a correction surgical procedure a suitable material for thread manufacturing is desired. In many cases, e.g., as described in the U.S. Pat. No. 3,123,077 in which a steel made thread is employed for this end; such material has a first advantageous feature: metallic materials, such as steel, has good features of mechanical strength—the best in the art—however, work time of the steel material in the Human body inner side is shorten in view of the stain process of the steel. Even stainless steel is able to be corrosion attacked. Other metallic materials like aluminum do not provide the desired features and may cause infectious processes in the inner side. In many cases, even the metals have good mechanical features, they are toxic for forming metallic complexes with many organic substances of the organism.

There is in the art a generation of plastic materials which are inert and have excellent mechanical properties. A non limiting example of such materials comprise nylon, kevlar, ethylene polymer, propylene polymers, and others in the art.

Another important feature related to the thread manufacturing comprise the arrangement of the lifting structures alongside the thread. In the art such structures are normally sting type (arrow point shape) and have a sharp edge. It is due to the fact that the lifted tissue is best supported in such manner.

The principal aim of the lifting threads of the invention is just to support the flaccid tissues which are able to be stung. The threads of the present invention provide tissue support in a novel fashion and totally free from the above discussed problems of the art: the arrangement of the support members provides a supporting condition comprising bristles having an intermediary level of softness. The characteristically soft flaccid tissue involves the contour of the bristles separately and also being placed inside the arrangement. Such feature permits that each arrangement be a fix point of the thread in the tissue and not just the bristle (or claws as mentioned in the art) separately; these bristles act as differential reinforcement members and they provide an fix interface of the thread and the tissue.

The idea of a fixing arrangement provides more than this functional feature: if defined from suitable parameters, the claws do not need to be arranged alongside the entire surface of the thread, but just in specific regions of it. This permits that a more simple and less expensive thread can be manufactured in comparison to the art.

Another matter related to the claws arrangements comprise fixing points which are placed locally; it can permit to reach aesthetical effects very similar to the natural condition of the human tissue, i.e., the movement done by the supported tissue are very similar to the corresponding natural movement and it makes the surgery correction practically invisible, the same is not possible from the surgical threads of the art. Known surgical threads has claws arrangements alongside the whole thread and it forces the tissue to do a simultaneous movement and the result is a tissue folding in the thread surroundings, and a non natural effect. The thread of the present invention consider the biomechanics of the dermal tissue, particularly soft tissues, permitting a balanced effect for make possible the tissue lifting and the tissue support in relation to the youth maker vectors and permitting natural foldings of the tissue in a very natural fashion.

Another solution provided by the present invention is a specific thread for any particular lifting surgery situation. No specific threads are seen in the art according to a particular lifting surgery necessity and considering the youth maker vectors mentioned above. The distribution of the soft tissue mass in a Human body comprise many cases, therefore the detection of a suitable thread profile for a particular ptosis is strongly desired. In response to this problem, threads of the present invention comprise different thread series and each kind of thread is particularly designed for a specific ptosis surgery correction.

According to the present invention support threads having particular structural features for any surgery situation are provided by means of an obtention method which is based on structural parameters of each model. The following comprise different sets of thread:

1—Simple Claw Thread

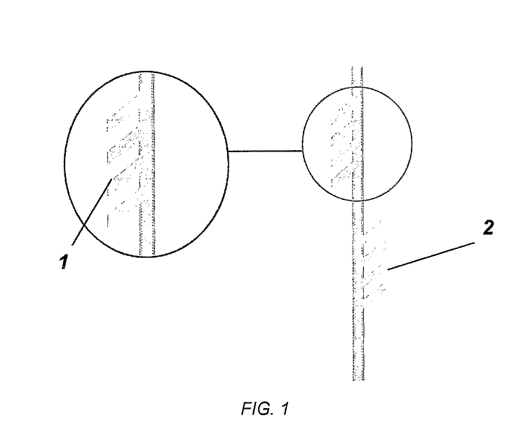

The simple claw thread, FIG. 1, comprises a not sequenced claw arrangement divided in fixing and traction claws which are placed in defined gaps. Such gaps are responsible for the natural movement effect of the surgical thread. The claws of the invention have rectangular shape and they are just called “claws” for correspond to the conventional name of the art.

As stated above, the arrangement of claws do not cause tissue stinging, conversely such claws provide an arrangement defining a new support interface between the thread and the tissue. The lines of claws are symmetrically opposed in relation to a central thread axis in which each claw of a claw line is out of phase in relation to the opposite claw.

The first upstream set (1)—called first set—of the simple claw arrangement is to perform a tissue traction and a supported tissue position while the second downstream set (2)—called second set—of simple claw is to support the tissue lifted by the first set. The definition of upstream and/or downstream is based on the traction direction of the thread. The enhancing of the thread area is to enhance the anchor effect of the thread in the face tissue, therefore when the thread is tractioned more tissue would be also tractioned.

If the thread would not have claws, no traction effect would be achieved and no tissue traction too. The present thread is manufactured based on the parameters of the Table 1.

TABLE 1
structural parameters for support thread manufacturing

| Parameter | Range |
|---|---|
| Lenght | 10 cm-50 cm |
| Central Axis Diameter | 0.20 mm-0.80 mm |
| Central Reinforcement Element | 1.0 mm-8.00 mm |
| Claw lenght | 26 mm-76 mm |
| Number of claws per set | 10-45 |
| Claw high | 15 mm-90 mm |
| Claw width | 10 mm-35 mm |
| Fixing angle of the claw in the thread | 10°-45° |
| Gap between claws | 0.5 mm-2.00 mm |

2—Conical Claw Thread

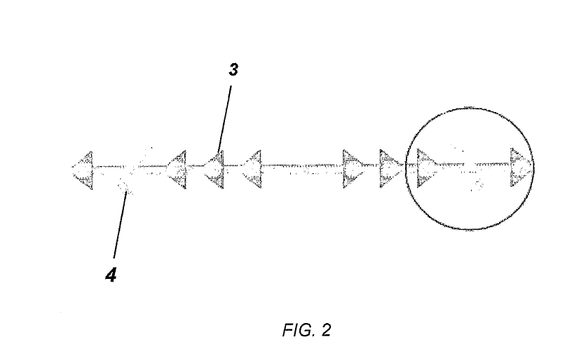

A thread of conical claws comprise another arrangement embodiment for surgical threads in which the claws rise from the central axis and comprise segments gap spaced one from the other in which a segment comprises two conical shaped claws altered by double-double claws (4). Each segment of upstream/downstream conical claws (3) comprise 8 conical elements as in FIG. 2 and in accordance to the Table 2. The conical arrangement element combined to the pair of simple claws—forming a set—are as in the previous discussed case.

The arrangements of this embodiment comprise opposed elements in order to each element be able to perform a traction operation when the other element performs a support operation.

TABLE 2
Structural Parameters for support thread manufacturing.

| Parameter | Range |
|---|---|
| Lenght | 10 cm-50 cm |
| Central Axis Diameter | 0.20 mm-0.80 mm |
| Central Reinforcement Element | 1.0 mm-8.00 mm |
| Claw free Terminal Segment | 10 mm-25 mm |
| Claw high | 0.30 mm-1.00 mm |
| Claw Diameter | 0.10 mm-2.00 mm |
| Claw Fixing angle in relation to the | 20°-90° |
| Central Axis | |
| Number of Claws per thread | 10-40 |

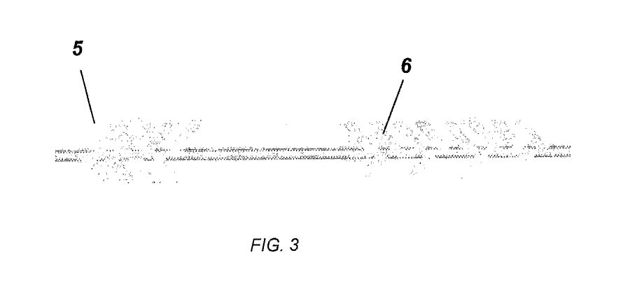

The double converged thread, FIG. 3, comprises an arrangement in which a first set of simple claws (5) are placed in alternate position and having a phase difference comprising a half period (i.e., π rad. delayed one in relation to the other) and such set is positioned in an opposite fashion in relation to a second set of simple claws (6). The second set of simple claws comprise a number of alternate positioned claws and having a 2,5 periods of phase delay (5π rad.). As in the above discussed cases, each set of claws is able to perform a traction operation while the other set is performing a support operation. The corresponding parameters are outlined in Table 3.

TABLE 3
Parameter for a support thread manufacturing.

| Parameter | Range |
|---|---|
| Lenght | 10 cm-50 cm |
| Number of claws per thread | 80-190 |
| Cross section lenght of the thread | 35 mm-90 mm |
| Gap between a claws pair | 1.10 mm-2.00 mm |
| Number of claws | 20-70 |
| Central Axis Diameter | 0.30 mm-1.00 mm |
| Claw high | 0.30 mm-1.00 mm |
| Claw width | 0.10 mm-0.50 mm |
| Claw fixing angle | 30°-45° |

4—Endoscopic Surgery Thread

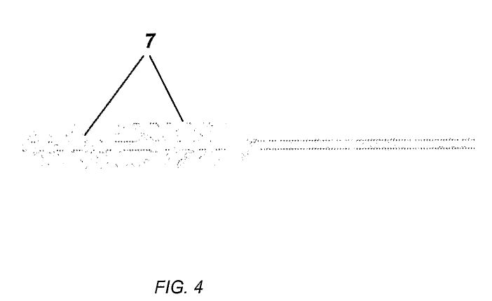

This thread (FIG. 4) comprises a rectangular shaped claw arrangement particularly the claws have are paralelogram shaped. It comprises double- 15 double claws (7) and each arrangement is spaced one from the other by a claw free thread portion having the same length of the arrangement. It means that this thread embodiment is in part covered by a set of double-double claws (7) and in part not. This thread is to perform tissue fixing, such as fat tissue fixing or Human muscular aponeurosis.

TABLE 4
Parameters for support thread manufacturing.

| Parameter | Range |
|---|---|
| Lenght | 10 cm-50 cm |
| Claw section lenght | 80 mm-190 mm |
| Claw free section lenght | 50 mm-120 mm |
| Distance between a pair of claws | 1.10 mm-2.00 mm |
| Number of claws | 20-70 |
| Central Axis Diameter | 0.30 mm-1.00 mm |
| Claw high | 0.30 mm-1.00 mm |
| Claw width | 0.10 mm-0.50 mm |
| Claw fixing angle | 30°-45° |
| Number of claws per thread | 20-100 |

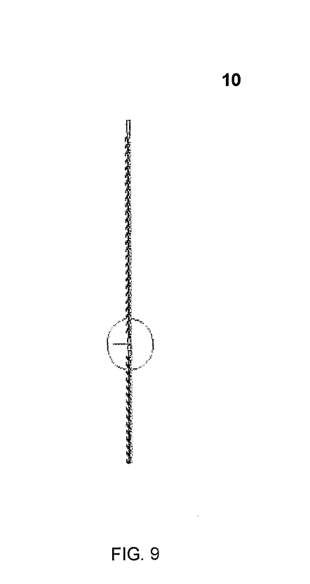

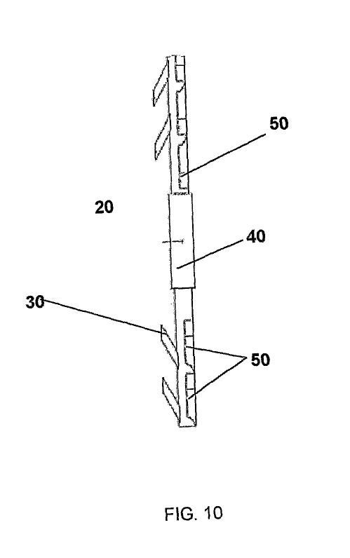

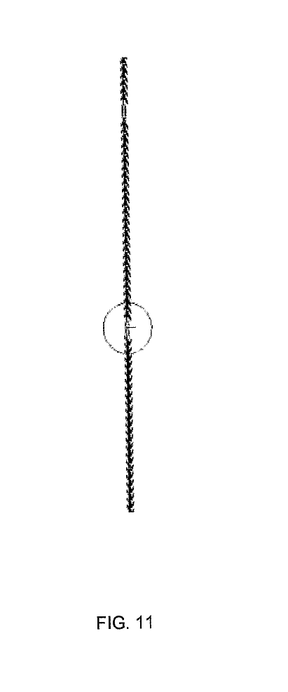

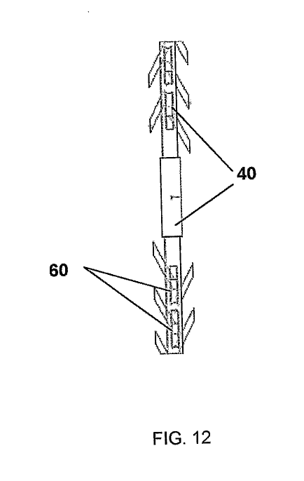

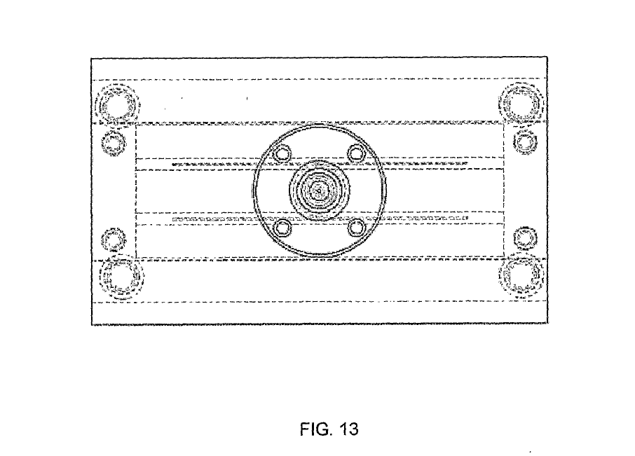

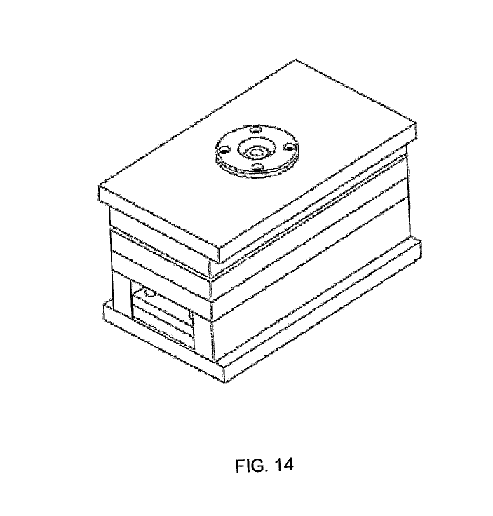

As seen in the above embodiments of the present invention, the surgical thread shall have a specific profile for a determined surgery purpose. In this sense, other embodiment can provided without depart of the scope of the present invention. A non limiting example of other embodiments comprise a surgical thread (10)—FIGS. 9 and 10—having a saw tooth type claw arrangement (30, 50) alongside the thread axis and each set of saw tooth type claws is spaced one from the other by a cilindrical body (40) as seen in the detailed view (20) of FIG. 10. Another preferred embodiment comprise the surgical thread of FIGS. 11 and 12. In this case, the surgical thread comprise a set of saw type claws (40, 60) radially placed in relation to the thread axis and do not having a common plane in which all claws of the thread are comprised.

The above mentioned threads just corresponds to some from the several surgical situations in which a thread having a specific arrangement is indicated in order to provide a lifting result having an effect as natural as possible.

For manufacture all these surgical threads, and many other if necessary, the present invention applied a moulding technique well known in the art. It comprise a moulding injection of a surgical thread wherein cavity of a metal plate is filled with a melt plastic material and the moulding equipment is kept under mechanical pressure during all these steps. All these procedure is easily found in the art.

The inventive step in this part of the present invention comprise the counter form of the mould cavity; the counter form comprises the design of the mould cavity inside which the melt plastic material will be injected. For an intended type of surgical thread having a particular arrangement of claws a mould cavity having the corresponding counter form is provided in order to obtain such thread. For example, for the above four cases of surgical thread having a particular claw arrangement a particular mould cavity having the corresponding counter form has been prepared.

Therefore in the case 1, SIMPLE CLAW THREAD, the respective counter form corresponds to an arrangement of not sequenced claws divided in fixing and traction claws having a paralelogram shape.

In the case 2, CONICAL CLAW THREAD, the respective counter form corresponds to an arrangement in which the claws rise from the central axis and comprise segments gap spaced one from the other in which a segment comprises two conical shaped claws altered by double-double claws (4). Each segment of upstream/downstream conical claws (3) comprise 8 conical elements.

In the case 3, DOUBLE CONVERGED THREAD, the respective counter form corresponds to an arrangement in which a first set of simple claws (5) are placed in alternate position and having a phase difference comprising a half period (i.e., π rad. delayed one in relation to the other) and such set is positioned in an opposite fashion in relation to a second set of simple claws (6) and the second set of simple claws comprise a number of alternate positioned claws and having a 2,5 periods phase delay (5π rad.).

In the case 4, ENDOSCOPIC SURGERY THREAD, the respective counter form corresponds to a paralelogram shaped claw arrangement comprising double-double claws (7) and each arrangement is spaced one from the other by a claw free thread portion having the same length of the arrangement.

Further, a mould cavity counter form corresponds to any suitable claw arrangement of a surgical thread of the present invention, for example other suitable counter form corresponds to one arrangement in which a saw tooth type claw arrangement alongside the thread axis is provided and each set of saw tooth type claws is spaced one from the other by a cilindrical body. Other suitable mould cavity counter form of the present invention corresponds to a saw type claws radially placed in relation to the thread axis and do not having a common plane in which all claws of the thread are comprised.

### EXAMPLES

In this part a set of examples will be presented in order to offer a better understanding of the present invention, but such examples are not limiting of the scope of the present invention.

1^{st} Polypropylene Eight Threads Arrangement (FP2C) for the Face Sides Including the Neck.

### Example 1

### Facial Lifting Surgery

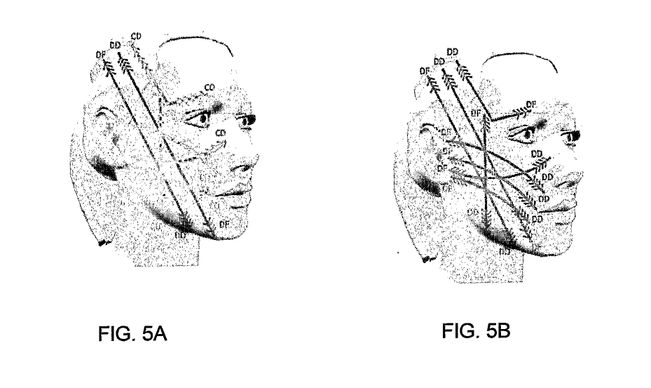

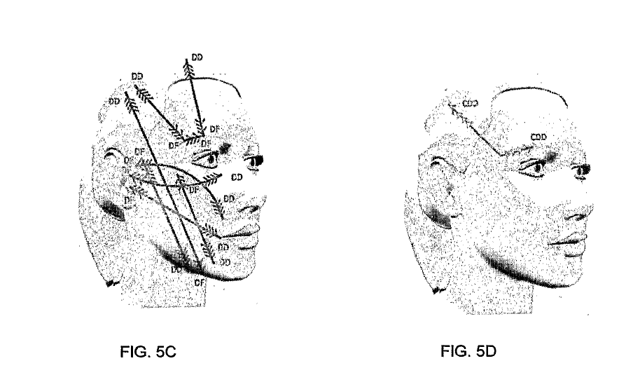

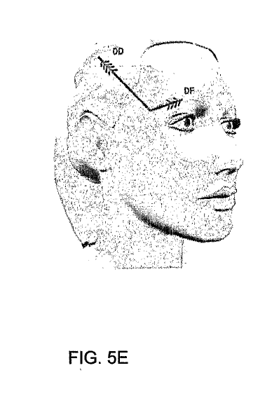

The method for insertion of the thread in the deep fat tissue involves eight FP2C type threads in each one of the face sides permitting that the ptosis tissues of the adipous structures can be positioned according to the youth maker vectors (FIG. 5). In view of the shape of the double-double and double-claw free claw set (identified as DD-DF and better seen in the FIG. 3) e and methodology for insertion in the deep adipous tissue by means of a helical torsion, the FC2P threads, also called maximum torsion threads, can lift and support the ptosis tissues returning them to their original position as natural as possible (natural tissue movement and aesthetical effect). The claws of the art, however, do not permit the same result due to such claws have a fragile structure for traction and support operations and they have a less functional feature in relation to the original position procedure of the tractioned tissue.

Other favorable feature of the FP2C threads from the threads of the art comprises the suitability of the former for ptose correction cases in specific regions providing a natural facial aspect. Depending on the region to be corrected, the insertion axis can be inverted for some FP2C threads in order to permit that some double-double claws can act as static fixing axis. This is applied when a mouth and eye brow correction surgery are necessary for obtain an effect as natural as possible. Also the Master Thread can have its axis inverted when an anchor effect in the soft tissues.

FIGS. 5A-5C illustrate, but not in a limiting way, three aesthetical facial lifting schemes. Basically, a set of threads in which a 20 cm length Master Thread having and a serie of traction thread having 12 cm length are provided. The Master Thread performs the central supporting of the tissue, providing a basic supporting structure over which the other threads complementarly act for provide a natural aspect of the tissue.

The number of threads may vary, depending on the extension of the flaccid tissue, ranging from 1 to 2 Master Threads, the same occurring with the complementary threads.

TABLE 5
Schemes for application of a set of aesthetical surgery
supporting threads

| | Types of thread | Number | |
|---|---|---|---|
| Case | and claw | of threads | Application |
| Scheme 1- | Maximum Traction | 1 Master Thread | Facial |
| | with claws DD-DF | (20 cm) and 7 | Aesthetic |
| | | Complementary | Surgery |
| | | threads | |
| Scheme 2- | Maximum Traction | 2 Master Threads | Facial |
| | Thread with DD- | (20 cm) and 5 | Aesthetic |
| | DF claws | Complementary | Surgery |
| | | Threads (12 cm) | |
| Scheme 3- | Maximum Traction | 2 Master Threads | Facial |
| | Thread with DD- | (20 cm) and 5 | Aesthetic |
| | DF claws and | Conical Threads | Surgery |
| | Total Traction | (12 cm) | |
| | Threads with | | |
| | conical claws (CD) | | |
| Eye brow support | Maximum Traction | One support | Facial |
| | Thread with CD | conical thread | Aesthetic |
| | claws | 12 cm | Surgery |
| Eye brow support | Maximum Traction | One Support | Facial |
| | Thread with DD- | conical thread of | Aesthetic |
| | DF claws | 12 cm | Surgery |

### Example 2

### Surgery for Facial Paralysis Correction

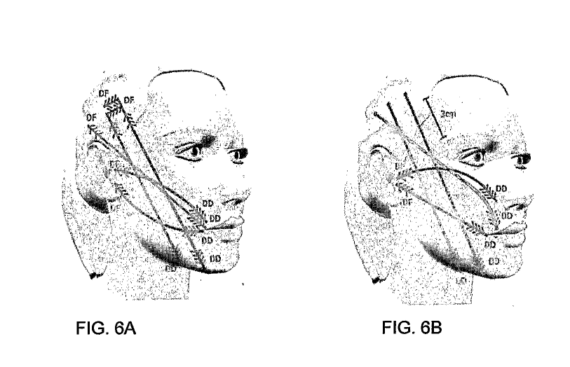

F2CP threads can be also used in cases of paralysis correction as seen in FIGS. 6A-6B. In such cases, the surgical approach comprises a variation of the surgical approach illustrated in FIG. 5. Master threads and complementary threads are employed in this case, however, due to the nature of the correction surgery involving handle type insert (FIG. 6A) and suture related implants (FIG. 6B).

TABLE 6
Scheme of support thread application for facial paralysis
correction surgery.

| | Types of Threads | | |
|---|---|---|---|
| Case | and Claws | Number of Claws | Application |
| Handle type | Maximum Traction | Three Master | Facial |
| implant | Thread with DD- | Threads (20 cm) | Paralysis |
| | DF claws | and two | Correction |
| | | Complementary | |
| | | Threads (12 cm) | |
| Suture | Maximum Traction | Two | Facial |
| Implants | Threads DD-DF | Complementary | Paralysis |
| | and Endoscopic | Threads (12 cm) | Correction |
| | Threads with DD- | and three | |
| | DF claws having | Endoscopic | |
| | claw free | Master Threads | |
| | segments | (20 cm) | |

### Example 3

### Aesthetical Surgery in the Neck Region

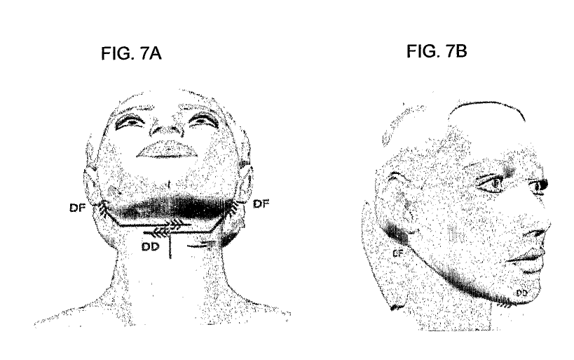

Threads can be advantageously applied in flaccid tissue support surgery in the neck region. In this case two 20 cm Master Threads symmetrically placed alongside the maxilar line toward the ears lobe are employed. Also, threads having DD-DF claws are employed (FIGS. 7A-7B).

TABLE 7
Scheme of Support thread application in neck falccid
tissues support surgery.

| | Types of threads with | |
|---|---|---|
| Case | claws | Application |
| Aesthetic support | Maximum Traction | Neck flaccid tissue |
| surgery. | Thread with DD-DF claws | support. |
| Aesthetic support | Maximum Traction | Neck flaccid tissue |
| surgery. | Thread with DD-DF claws | support. |

### Example 4

### Additional Application of the First Arrangement: Diasthasys Correction Surgery in the Arms and Umbilicus Lifting Surgery.

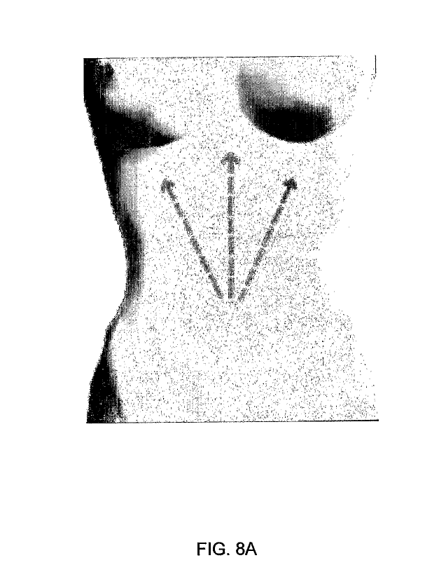

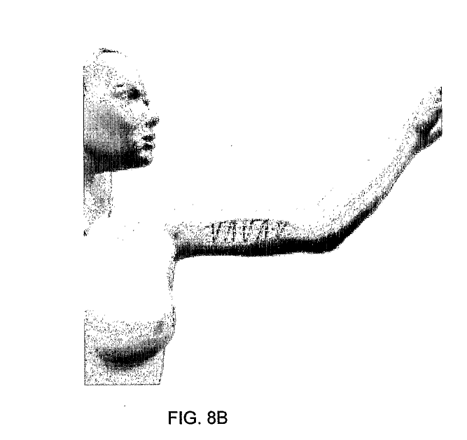

The FP2C thread is also employed in arm diasthasys correction and in umbilicus lifting surgery as in FIGS. 8A-8B. In the latter case, Maximum Traction Threads comprising 3 Master Threads with DD-DF claws are employed (FIG. 3).

TABLE 8
Scheme of Support thread application in arm diasthasys
correction surgery and in umbilicus lifting.

| | Types of Threads | Number | |
|---|---|---|---|
| Case | with claws | of threads | Application |
| Arm flaccid tissue | Maximum Traction | 4-5 Maximum | Arm |
| support | Thread with DD- | Traction Threads | diasthasys |
| | DF claws | (12 cm) | Correction |
| Abdoment flaccid | Maximum Traction | Three Master | Umbilicus |
| tissue support | Thread with DD- | Threads (20 cm) | lifting |
| | DF claws | | |

2^{nd} Arrangement—Simple Type Thread

This type of thread is employed in thin faces having a minimum of adipous tissue; the claw arrangement comprise simple claws in a convergent system. It is also indicated for combined applications when two FP2C are employed (Master Threads having 20 cm each one) for cases of facial flaccid cutaneous tissues. Six threads are employed in each side of the face.

3^{rd} Arrangement—Conical Threads

Due to the traction and total support strength, combined FP2C with 20 cm, Master Threads are employed for position ptosis suffered facial tissues having extremely flaccid volumous adipous tissue. Commonly, five conical threads in each side of the face and two parallel implants of FP2C having 20 cm are employed. Also, the conical threads are being used joined the FP2C in ptosis cases located in the facial middle third part and in the lower third part of the face, and also in cases of facial paralysis correction.

4^{th} Arrangement—Endoscopic Thread (Facial Paralysis Correction)

This thread has particularly comprises DD-DD (double-double) claws having opposed directions and comprising 10 cm of the thread total area while the other half part, 10 cm, is claw free. The claw free part is fixed in the aponeurosis or in the muscle by means of endoscopic procedures aiming to correct the tissue ptosis of the soft tissues for any kind of facial paralysis.

The toothed segment is inserted in the deep adipous tissue in order to position such tissue and correct the ptosis caused by the facial paralysis. Two FP2C threads having 12 cm of length are employed as a reinforcement support of the ptosis suffered tissue for a facial symmetry recovery of the paralized tissue in relation to the corresponding other side of the face.

1. Surgical thread employed in aesthetic and correction surgeries for support of flaccid motion limited tissues comprising a previous defined sequence of tissue support arrangement in which the arrangement comprise a number of fixing claws placed alongside of the thread surface.

2. Surgical thread according to claim 1, comprising a first arrangement of an upstream claw (1) set and a downstream claw (2) set.

3. Surgical thread, according to the preceeding claims, comprising a thread length ranging from 10 cm to 50 cm.

4. Surgical thread, according to claims 1 and 2, comprising a thread central axis diameter ranging from 0.2 mm and 0.80 mm.

5. Surgical thread, according to claims 1 and 2 comprising a thread central reinforcement element ranging from 1.0 mm e 8.00 mm.

6. Surgical thread according to claims 1 and 2 comprising a claw distribution ranging from 26 mm and 76 mm alongside the thread axis.

7. Surgical thread, according to claims 1 and 2, comprising a number of claws ranging from 10 to 45.

8. Surgical thread, according to claims 1 and 2, comprising a claw high ranging from 15 mm and 50 mm.

9. Surgical thread, according to any one of claims 1 and 2 comprising a claw width ranging from 10 mm and 35 mm.

10. Surgical thread, according to claims 1 and 2, comprising a fixing angle ranging from 10° and 45°.

11. Surgical thread, according to claims 1 and 2 comprising a claw distance ranging from 0.5 mm and 2.00 mm.

12. Surgical thread, according to claim 1, comprising a second arrangement of three conical claws (3) in an alternate fashion in relation to a pair of double-double claws (4).

13. Surgical thread, according to claim 12, wherein the alternate fashion of the claws define an upstream set of claws and a downstream set of claws which are symmetrically opposed.

14. Surgical thread, according to the claims 12 and 13 comprising a length ranging from 10 cm to 50 cm.

15. Surgical thread, according to claims 12 and 13 comprising a thread central diameter ranging from 0.20 mm to 0.80 mm.

16. Surgical thread, according to claims 12 and 13, comprising a thread central reinforcement element ranging from 1.0 mm to 8.00 mm.

17. Surgical thread, according to claims 12 and 13 comprising a claw free thread terminal segment ranging from 10 mm to 25 mm.

18. Surgical thread, according to claims 12 and 13, comprising a claw high ranging from 0.30 mm to 1.00 mm.

19. Surgical thread, according to claims 12 and 13 comprising a claw diameter ranging from 0.10 mm to 2.0 mm.

20. Surgical thread, according to claims 12 and 13, comprising a claw fixing angle in relation to the thread central axis ranging from 20° to 90°.

21. Surgical thread, according to claims 12 and 13 comprising a number of claws ranging from 10 to 40.

22. Surgical thread, according to claim 1 comprising a third arrangement containing a first set of simple claws (5) positioned in an alternate fashion having a phase delay related to consecutive claws of ½ period.

23. Surgical thread, according to claim 22, wherein the first set of simple claws (5) is positioned in an opposed fashion in relation to a second set of simple claws (6).

24. Surgical thread, according to claim 23, wherein the second set of simple claws (6) comprise a sequence of claws in an alternate fashion having a phase delay of 2½ periods.

25. Surgical thread, according to claims 22, 23 and 24, comprising a thread length ranging from 10 cm to 50 cm.

26. Surgical thread, according to claims 22, 23 and 24 comprising a claw total number ranging from 80 to 190.

27. Surgical thread, according to claims 22, 23 and 24 comprising a thread section length ranging from 35 mm to 90 mm.

28. Surgical thread, according to claims 22, 23 and 24, comprising a claw distance ranging from 1.10 mm and 2.0 mm.

29. Surgical thread, according to claims 22, 23 and 24 comprising a number of claws ranging from 20 and 70 claws.

30. Surgical thread, according to claims 22, 23, and 24 comprising a central axis diameter ranging from 0.3 mm to 1.00mm.

31. Surgical thread according to claims 22, 23 and 24 comprising a claw width ranging from 0.10 mm to 0.50 mm.

32. Surgical thread, according to claims 22, 23 and 24 comprising a claw high ranging from 0.30 mm to 1.00 mm.

33. Surgical thread, according to claims 22, 23 and 24 comprising a fixing angle ranging from 30° and 45°.

34. Surgical thread, according to claim 1, comprising a fourth claw arrangement wherein a set of double-double claws (7) are positioned in an alternate fashion in relation to claw free segments having the same length of the claw containing segment.

35. Surgical thread, according to claim 34, comprising a thread length ranging from 10 cm to 50 cm.

36. Surgical thread, according to claim 34 comprising a claw containing segment ranging from 80 mm to 190 mm.

37. Surgical thread, according to claim 34 comprising a claw free segment ranging from 50 mm to 120 mm.

38. Surgical thread, according to claim 34, comprising a distance between claws ranging from 1.10 mm and 2.00 mm.

39. Surgical thread, according to claim 34 comprising a number of claws ranging from 20 to 70.

40. Surgical thread, according to claim 34, comprising a thread central axis diameter ranging from 0.30 mm to 1.00 mm.

41. Surgical thread, according to claim 34, comprising a claw high ranging from 0.30 mm to 1.00 mm.

42. Surgical thread, according to claim 34 comprising a claw width ranging from 0.10 mm to 0.50 mm.

43. Surgical thread, according to claim 34 comprising a fixing angle ranging from 30° and 45°.

44. Claw arrangement for a surgical thread employed in aesthetical and correction surgeries for flaccid tissue support ou tissues having motion limitation comprising a first set of upstream claws (1) and a second set of downstream claws (2) defining a simple claws thread.

45. Claw arrangement for a surgical thread employed in aesthetical and correction surgeries for flaccid tissue support ou tissues having motion limitation comprising three conical claws (3) in an alternate fashion in relation to a pair do double-double claws (4) defining a conical element thread.

46. Claw arrangement for a surgical thread employed in aesthetical and correction surgeries for flaccid tissue support ou tissues having motion limitation comprising a first set of simple claws (5), placed in an alternate fashion and having a 2½ periods phase delay one in relation to the other; the first set of simple claws (5) is in an opposite fashion in relation to a second set of simple claws (6) which are also in an alternate fashion and having a 2½ periods phase delay one in relation to the other; the first set of simple claws (5) and the second set of simple claws (6) define a double converged thread.

47. Claw arrangement for a surgical thread employed in aesthetical and correction surgeries for flaccid tissue support ou tissues having motion limitation comprising a fourth arrangement of double-double claws set (7) in an alternate fashion in relation to claw free segment distributed in the same extension of the claw containing segment for define an endoscopic surgery thread.

48. Claw arrangement for a surgical thread employed in aesthetical and correction surgeries for flaccid tissue support ou tissues having motion limitation comprising a saw tooth type claw arrangement (30, 50) placed alongside the thread axis and each set of saw tooth type claws being spaced one from the other by a cylindrical body (40).

49. Claw arrangement for a surgical thread employed in aesthetical and correction surgeries for flaccid tissue support ou tissues having motion limitation comprising saw type claws (40, 60) arrangement radially placed in relation to the thread axis and do not having a common plane in which all claws of the thread are comprised.

50. Injection mould having a moulding cavity wherein a simple claw thread is defined by the respective counter form of the injection mould cavity corresponding to an arrangement of not sequenced claws divided in fixing and traction claws having a paralelogram shape.

51. Injection mould having a moulding cavity wherein a conical claw thread is defined by the respective counter form of the injection mould cavity corresponding to an arrangement in which the claws rise from the central axis and comprise segments gap spaced one from the other in which a segment comprises two conical shaped claws altered by double-double claws (4); and each segment of upstream/downstream conical claws (3) comprise 8 conical elements.

52. Injection mould having a moulding cavity wherein a double converged thread is defined by the respective counter form corresponding to an arrangement in which a first set of simple claws (5) are placed in alternate position and having a phase difference of ½ period which comprises a π rad. phase delay one in relation to the other and such set being positioned in an opposite fashion in relation to a second set of simple claws (6) and the second set of simple claws comprise a number of alternate positioned claws and having a phase difference of 2½ periods which comprises a phase delay of 5π rad.

53. Injection mould having a moulding cavity wherein a endoscopic surgery thread is defined by the respective counter form corresponding to a paralelogram shaped claw arrangement comprising double-double claws (7) and each arrangement is spaced one from the other by a claw free thread segment having the same length of the claw containing segment.

54. Injection mould having a moulding cavity wherein a saw tooth type thread is defined by the corresponding counter form corresponding to a saw tooth claw arrangement in which each set of saw tooth type claws is spaced one from the other by a cylindrical body.

55. Injection mould having a moulding cavity wherein a saw tooth type thread is defined by the respective counter form corresponding to a saw tooth claw arrangement comprising saw type claws radially placed in relation to the thread axis and do not having a common plane in which all claws of the thread are comprised.

56. Use of surgical threads for aesthetical and correction surgeries of flaccid tissue support or motion limited tissue correction wherein one or more surgical threads in accordance to claims 1 to 43 are employed.

57. Use of surgical threads for aesthetical and correction surgeries of flaccid tissue support or motion limited tissue correction wherein, the surgical threads in accordance to the claims 1 to 43 comprise at least one claw arrangement in accordance to claims 44 to 49.
